(19) United States
(12) Patent Application Publication
(10) Pub. No.: US 2012/0093765 A1
(43) Pub. Date: Apr. 19, 2012

(54) PROCESS FOR PURIFICATION OF RECOMBINANT HUMAN GRANULOCYTE COLONY STIMULATING FACTOR

(75) Inventors: Sandeep Somani, Pune (IN); Sriram Padmanabhan, Pune (IN)

(73) Assignee: LUPIN LIMITED, Mumbai, Maharashtra (IN)

(21) Appl. No.: 13/378,973

(22) PCT Filed: Jun. 7, 2010
(86) PCT No.: PCT/IN2010/000377

§ 371 Date: Dec. 16, 2011

(30) Foreign Application Priority Data
Jun. 16, 2009 (IN) 865/KOL/2009

(51) Int. Cl. A61K 38/19; C07K 1/36; C07K 14/535; C07K 1/14

(52) U.S. Cl. 424/85.1; 530/395

(57) ABSTRACT

The present invention describes a novel process for large-scale purification of therapeutic grade quality of recombinant human GCSF from microbial cells, wherein the protein is expressed as inclusion bodies. The Inclusion bodies are solubilized and refolded under redox condition. The Redox condition is provided by using ascorbic acid, dehydroascorbic acid and reduced gluthathione. The process involves the novel use of aqueous two phase extraction step to purify refolded GCSF after removal of denaturant. After this step GCSF is further purified using chromatography techniques for removal of related impurities. The GCSF obtained has good purity and yields which are essential for a production scale process. The host cell related contaminants like proteins, DNA and endotoxins are reduced using the purification processes of the invention.

### FIELD OF THE INVENTION

The invention is related to process for purification of colony stimulating factors using at least one step of aqueous two phase extraction process. Particularly the invention is related to the process for the purification of the recombinant human GCSF using aqueous two phase extraction process. The invention is also related to purified recombinant human GCSF produced by the processes of the invention resulting in lesser oxidative forms, endotoxins and host cell proteins.

### BACKGROUND OF THE INVENTION

Colony-stimulating factors (CSFs) are secreted glycoproteins which bind to receptor proteins on the surfaces of hemopoietic stem cells and thereby activate intracellular signaling pathways which can cause the cells to proliferate and differentiate into a specific kind of blood cell. Human granulocyte-colony stimulating factor (h-GCSF) and human macrophage granulocyte-colony stimulating factor (h-GM-CSF) belongs to a group of colony stimulating factors that play an important role in stimulating the differentiation and proliferation of hematopoietic precursor cells and activation of mature neutrophils. GCSF is capable of supporting neutrophil proliferation in vitro and in vivo. GCSF protein has only one single O-glycosylation site at threonine 133; absence of glycosylation at this residue was not found to affect the stability of the protein. For many protein therapeutics where glycosylation of the protein is known to affect stability, it is necessary to undertake cloning and expression in yeast or mammalian cells, using appropriate expression vectors. In the case of GCSF, the recombinant protein expressed in E. coli was found to have the same specific activity as the native protein (Oh-eda et. al. 1990 J. Biol. Chem. 256,11432-11435, Hill et. al. 1993 Proc. Nat. Acad. Sci. USA 90.5167-5171, and Arakawa et. al. 1993 J. Protein Chem. 12, 525-531). Human GCSF in its naturally occurring form is a glycoprotein having a molecular weight of about 20,000 Dalton and five cysteine residues. Four of these residues form two intramolecular disulfide bridges which are of essential importance for the activity of the protein. As GCSF is available only in small amounts from its natural sources, recombinant forms of GCSF are mainly used for producing pharmaceuticals, which can for example be obtained by means of expression in mammalian cells like CHO (Chinese Hamster Ovary) cells or in prokaryotic cells like E. coli. The recombinant proteins expressed in mammalian cells differ from naturally occurring GCSF in that they have a different glycosylation pattern, while in the proteins expressed in E. coli which can have an additional N-terminal methionine residue as a result of bacterial expression, glycosylation is not present at all. The cloning and expression of cDNA encoding human GCSF has been described by two groups (Nagata, S. et. al., Nature 319, 415-418 (1986); Souza, L. M. et al., Science 232, 61-65 (1986)).

The recombinant production of GCSF has been described in patent literature for the first time in 1987, in WO 87/01132 A1. The first commercially available GCSF is produced and distributed by Amgen under the trade name Neupogen(R). While the production of GCSF in prokaryotic cells is preferred as compared to the production in mammalian cells, as the use of simpler expression systems and culture conditions is possible. However a frequently occurring problem in the production of recombinant proteins in prokaryotic cells is, the formation of hardly soluble intracellular aggregates of denatured forms of the protein expressed called as inclusion bodies, which partially have a secondary structure and can be found in the cytoplasm of the bacterial cells. The formation of said inclusion bodies leads to the necessity of solubilizing and renaturing the proteins subsequent to the isolation of the inclusion bodies by means of centrifugation at moderate speed with the aid of suitable means in order to maintain their active configuration. Herein, the competitive reaction between a transfer of the denatured protein into the right folding intermediate and an aggregation of several protein molecules is an essential factor limiting the yield of renatured protein.

Many earlier patents have described various aspects of recombinant expression and purification of the GCSF protein from different expression systems ranging from bacterial cells to yeast and mammalian cells. Some of the processes described are multi-step processes where losses in yield at the end of the purification process can be significant. The following U.S. Pat. Nos. 4,810,643; 4,999,291; 5,582,823; 5,580,755; and 5,830,705, and PCT publications WO 87/03689, WO 87/02060, WO 86/04605 and WO 86/04506 describe various aspects of recombinant expression and purification of the h-GCSF protein from various expression systems ranging from bacterial cells to yeast and mammalian cells.

Various other methods have been reported in scientific literature for the purification of GCSF expressed in E. coli, yeast or CHO cells. A method of purification of GCSF from CHU-2 conditioned medium (human oral carcinoma cell line), which is known to produce GCSF constitutively was developed by Nomura et. al. (EMBO J. vol 5,871, 1986). The process describes the use of a three-step chromatography procedure after concentration and ultrafiltration of the conditioned medium. WO 87/01132 A1 describes the cation exchange chromatographic purification of GCSF.

Purification of GCSF in bacterial systems is disclosed in U.S. Pat. Nos. 4,810,643 and 4,999,291. Several chromatographic based purification of GCSF has been described in the prior art for example PCT publication Nos. WO 03/051922 A1, WO 01/04154 A1,

U.S. Pat. No. 5,055,555, describes a simplified process for purification of recombinant hGCSF expressed from eukaryotic cells. After ion exchange chromatography the protein is precipitated by salting our using sodium chloride. But for recovery of GCSF from inclusion bodies expressed in bacteria, precipitation of the protein by sodium chloride salt, increases the aggregation status resulting in loss of yield and activity

The various purification protocols discussed in the above patents mention multiple chromatography and other steps for the purification of GCSF. None of the above literature disclosed a simple and viable processing method for the production of pharmaceutical grade GCSF on industrial scale.

Purification technique known as aqueous two-phase extraction was introduced in 1956-1958 with applications for both cell particles and proteins. Since then, it has been applied to a host of different materials, such as plant and animal cells, microorganisms, viruses, chloroplasts, mitochondria, membrane vesicles, proteins, and nucleic acids. The basis for extraction by a two-phase system is selective distribution of substances between the phases. For a soluble substance, distribution occurs mainly between the two bulk phases, and the extraction is characterized by the partition coefficient, which is defined as the concentration of partitioned substance in the top phase, divided by the concentration of the partitioned substance in the bottom phase. Ideally, the partition coefficient is independent of total concentration and the volume ratio of the phases. It is mainly a function of the properties of the two phases, the partitioned substance, and the temperature. The two-phase systems may be produced by mixing two phase-incompatible polymer solutions, by mixing a polymer solution and a salt solution, or by mixing a salt solution and a slightly apolar solvent. These types of systems, along with aqueous two-phase extraction methods for separating macromolecules such as proteins and nucleic acids, cell particles, and intact cells are described in the literature, for example, in Albertsson, Partition of Cell Particles and Macromolecules, 3rd edition (John Wiley & Sons: New York, 1986); Walter et al., Partitioning in Aqueous Two-Phase Systems: Theory, Methods, Uses, and Applications to Biotechnology, (Academic Press: London, 1985).

Several low-cost two-phase systems are known that can handle protein separations on a large scale. These systems use polyethylene glycol (PEG) as the upper phase-forming polymer and crude dextran (e.g., Kroner et al., Biotechnology Bioengineering, 24:1015-1045 [1982]), a concentrated salt solution (e.g., Kula et al., Adv. Biochem. Bioeng., 24: 73-118 [1982]), or hydroxypropyl starch (Tjerneld et al., Biotechnology Bioengineering, 3.0:809-816[1987]) as the lower phase-forming polymer.

Two-phase aqueous polymer systems are extensively discussed in the literature. See, e.g., Baskir et al., Macromolecules, 20: 1300-1311 (1987); Birkenmeier et al., J. Chromatogr., 360:193-201 (1986); Birkenmeier and Kopperschlaeger, J. Biotechnol., 21:93-108 (1991); Blomquist and Albertsson, J. Chromatogr., 73: 125-133 (1972); Blomquist et al., Acta Chem. Scand., 29: 838-842 (1975); Erlanson-Albertsson, Biochim. Biophys. Acta, 617: 371-382 (1980); Foster and Herr, Biol. Reprod., 46: 981-990 (1992); Glossmann and Gips, Naunyn. Schmiedebergs Arch. Pharmacol., 282: 439-444 (1974); Hattori and Iwasaki, J. Biochem. (Tokyo), 88: 725-736 (1980); Haynes et al., AICHE Journal-American Institute of Chemical Engineers, 37: 1401-1409 (1991); Johansson et al., J. Chromatogr., 331: 11-21 (1985); Johansson et al., J. Chromatogr., 331: 11-21 (1985); Kessel and McElhinney, Mol. Pharmacol., 14: 1121-1129 (1978); Kowalczyk and Bandurski, Biochemical Journal, 279: 509-514 (1991); Ku et al., Biotechnol. Bioeng., 33: 1081-1088 (1989); Kuboi et al., Kagaku Kogaku Ronbunshu, 16: 1053-1059 (1990); Kuboi et al., Kagaku Kogaku Ronbunshu, 16: 755-762 (1990); Kuboi et al., Kagaku Kogaku Ronbunshu, 17: 67-74 (1991); Kuboi et al., Kagaku Kogaku Ronbunshu, 16: 772-779 (1990); Lillehoj and Malik, Adv. Biochem. Eng. Biotechnol., 40: 19-71 (1989); Mattiasson and Kaul, “Use of aqueous two-phase systems for recovery and purification in biotechnology” (conference paper), 314, Separ. Recovery Purif.: Math. Model., 78-92 (1986); Ohlsson et al., Nucl. Acids Res., 5: 583-590 (1978); Wang et al., J. Chem. Engineering of Japan, 25: 134-139 (1992); Zaslayskii et al., J. Chrom., 439: 267-281 (1988); Zaslayskii et al., J. Chem. Soc., Faraday Trans., 87:141-145 (1991); U.S. Pat. No. 4,879,234 issued Nov. 7, 1989 (equivalent to EP 210,532); DD (German) 298,424 published Feb. 20, 1992; WO 92/07868 published May 14, 1992; and U.S. Pat. No. 5,093,254. See also Hejnaes et al., Protein Engineering, 5: 797-806 (1992).

An aqueous two-phase extraction/isolation system is described by DE 288,837. In this process for selective enrichment of recombinant proteins, a protein-containing homogenate is suspended in an aqueous two-phase system consisting of PEG and polyvinyl alcohol as phase-incompatible polymers. Purification of interferon has been achieved by selective distribution of crude interferon solutions in aqueous PEG-dextran systems or PEG-salt systems using various PEG derivatives as disclosed in German Patent DE 2,943,016.

U.S. Pat. No. 5,695,958 provides a method for isolating an exogenous polypeptide in a non-native conformation from cells, such as an aqueous fermentation broth, in which it is prepared comprising contacting the polypeptide with a chaotropic agent, preferably a reducing agent and with phase-forming species to form multiple aqueous phases, with one of the phases being enriched in the polypeptide which is depleted in the biomass solids and nucleic acids originating from the cells.

U.S. Pat. No. 6,437,101 describes the methods for the isolation of human growth hormone, growth hormone antagonist, or a homologue of either, from a biological source. The methods described in the '101 patent use multi-phase extraction process.

U.S. Pat. No. 7,060,669 provides processes for extraction of proteins of interest in aqueous two phase extraction by fusing said proteins to targeting proteins which have the ability of carrying said protein into one of the phases.

The main benefits of the extraction technique are the method is efficient, easy to scale up, rapid when used with continuous centrifugal separators, relatively low in cost, and high in water content to maximize biocompatibility. Currently there are relatively few industrial applications of aqueous two-phase system for purifying proteins.

Purification of GCSF protein in its native form and absence of denaturant using aqueous two phase extraction has not been described so far.

### SUMMARY OF THE INVENTION

In one aspect the invention is related to a process for the purification of recombinant human GCSF obtained in the form of inclusion bodies from microbial cells, which comprises at least one step of aqueous two phase extraction.

In another aspect the invention is related to a process for the purification of recombinant human GCSF obtained in the form of inclusion bodies from microbial cells, the process comprises the steps:
a) solubilizing the inclusion bodies of GCSF;
b) refolding the said solubilized GCSF proteins;
c) purifying the refolded GCSF by using aqueous two phase extraction;
d) optionally further purifying the native GCSF obtained in step c; and
e) isolating pure GCSF.

In another aspect the invention is related to the aqueous two phase extraction process for isolating native form of GCSF.

Another aspect of the invention is the purified GCSF obtained by the process of the invention comprising at least one step of aqueous two phase extraction process.

In further aspect the invention is related to the aqueous two phase extraction process for separating more than 95% of the host cell proteins, endotoxins and DNA from the refolded protein GCSF in the lower phase wherein the refolded protein is a mammalian polypeptide, (polypeptide that were originally derived from mammalian organism) that are expressed in the form of inclusion bodies in prokaryotic cells. This process could also be applied to GCSF purification from natural sources such as tissues and blood samples.

In another aspect the invention also relates to pharmaceutical composition comprising therapeutically effective amount of the biologically active GCSF obtained according to the process of the present invention comprising at least one step of aqueous two phase extraction process.

The details of one or more embodiments of the inventions are set forth in the description below. Other features, objects and advantages of the inventions will be apparent from the description and claims.

### BRIEF DESCRIPTION OF THE DRAWINGS

FIG. 1: Schematic Description of the aqueous two phase extraction process for purification of GCSF.

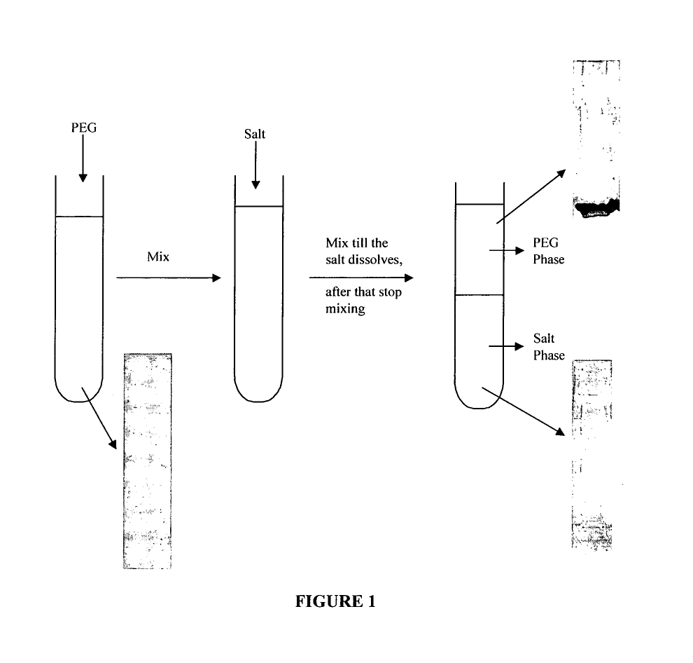

FIG. 2: SDS-PAGE profile of purification

### DETAILED DESCRIPTION OF THE INVENTION

As used herein, “reducing agent” refers to a compound that, in a suitable concentration in aqueous solution, maintains sulfhydryl groups so that the intra- or intermolecular disulfide bonds are chemically disrupted. Representative examples of suitable reducing agents include dithiothreitol (DTT), dithioerythritol (DTE), beta-mercaptoethanol (BME), cysteine, cysteamine, thioglycolate, glutathione, and sodium borohydride.

As used herein, “chaotropic agent” refers to a compound that, in a suitable concentration in aqueous solution, is capable of changing the spatial configuration or conformation of polypeptides through alterations at the surface thereof so as to render the polypeptide soluble in the aqueous medium. The alterations may occur by changing, e.g., the state of hydration, the solvent environment, or the solvent-surface interaction. The concentration of chaotropic agent will directly affect its strength and effectiveness. A strongly denaturing chaotropic solution contains a chaotropic agent in large concentrations which, in solution, will effectively unfold a polypeptide present in the solution. The unfolding will be relatively extensive, but reversible. A moderately denaturing chaotropic solution contains a chaotropic agent which, in sufficient concentrations in solution, permits partial folding of a polypeptide from whatever contorted conformation the polypeptide has assumed through intermediates soluble in the solution, into the spatial conformation in which it finds itself when operating in its active form under endogenous or homologous physiological conditions. Examples of chaotropic agents include guanidine hydrochloride, urea, and hydroxides such as sodium or potassium hydroxide. Chaotropic agents include a combination of these reagents, such as a mixture of base with urea or guanidine hydrochloride.

As used herein, the term “inclusion bodies” refers to dense intracellular masses of aggregated polypeptide of interest, which constitute a significant portion of the total cell protein, including all cell components. These aggregated polypeptides may be incorrectly folded or partially correctly folded proteins. In some cases, but not all cases, these aggregates of polypeptide may be recognized as bright spots visible within the enclosure of the cells under a phase contrast microscope at magnifications down to 1000 fold.

The term “therapeutically effective amount” used herein refers to the amount of biologically active G-CSF which has the therapeutic effect of biologically active G-CSF.

The term “biologically active G-CSF” used herein refers to G-CSF which is capable of promoting the differentiation and proliferation of hematopoietic precurser cells and the activation of mature cells of the hematopoietic system.

In an embodiment the invention provides a process for large scale purification of recombinant GCSF in native form obtained from microbial cells.

The process according to the present invention comprises the steps of:
a) solubilizing the inclusion bodies of GCSF;
b) refolding the said solubilized GCSF proteins;
c) purifying the refolded GCSF by using aqueous two phase extraction;
d) optionally further purifying the native GCSF obtained in step c; and
e) isolating pure GCSF.

According to one embodiment of the invention the inclusion bodies are dissolved in a suitable solublizing buffer and a suitable chaotropic agent at a pH in the range of 7 to 12. The suitable buffer includes but not limited to Tris (chloride/maleate) buffer, phosphate (sodium and potassium) buffer, glycine sodium hydroxide buffer, boric acid-borax buffer, borax-sodium hydroxide buffer, carbonate-bicarbonate buffer etc

The suitable chaotropic agents include urea and salts of guanidine or thiocyanate, preferably urea, guanidine hydrochloride, or sodium thiocyanate. The amount of chaotropic agent necessary to be present in the buffer depends, for example, on the type of chaotropic agent and polypeptide present. The amount of chaotropic agent required should be sufficient to unfold a polypeptide present in the solution. The pH of the solution will depend on the chaotropic agent, for urea the pH of the solution is maintained in the range of 9 to 12, for guanidine hydrochloride the pH is in the range of 7 to 9. The OD of the solution is in the range of about 2 to about 12.

The surfactants and other agents that could be used for soulubilizing microbial inclusion bodies include SDS, CTAB, CHAPS, Tween 20, Triton X100, Sarcosyl, Octyl betaglucoside, Nonidet P-40, dodecyl maltoside, NDSB.

(From ref: Process Scale Bioseparations for the biopharmaceutical industry, Ed by Abhinav A Shukla, Mark R Etzel and Shishir Gadam, Taylor and Francis, 2007 page 129, which is incorporated herein by reference in its entirety).

The solution containing solubilised inclusion bodies is treated with a reducing agent at a temperature in the range of 10 to 30° C. The reducing agent includes one or more of the dithiothreitol (DTT), betamercaptoethanol (BME); cysteine, thioglycolate, and sodium borohydride. The amount of reducing agent to be present in the buffer will depend mainly on the type of reducing agent and chaotropic agent, the type and pH of the buffer employed, and the type and concentration of the polypeptide in the buffer. An effective amount of reducing agent is that which is sufficient to eliminate intermolecular disulfide-mediated aggregation. The preferred reducing agent is DTT.

In an embodiment of the invention the protein GCSF is obtained in the native form by refolding the solubilized GCSF in the refolding buffer. Typically a refolding buffer may contain a suitable buffer, an amino acid such as arginine or proline, sucrose, EDTA, sodium ascorbate, urea. When sodium ascorbate is used in refolding buffer dehydro ascorbate and reduced glutathione are also added in refolding buffer to provide redox condition while refolding. Alternately oxido-shuffling agents such as Cysteine/Cystine or dxidised and reduced glutathione can also be used.

The refolding is carried out at a temperature in the range of 5 to 20° C., preferably at temperature of 6 to 10° C. The time required for the refolding may take from about 6 to 24 hrs, preferably between 15 to 20 hrs.

After the refolding of the protein is complete diafiltration may be performed. For the removal of the denaturant a buffer exchange may be carried out by using Tris buffer having sucrose or sorbitol.

In an embodiment of the invention the protein GCSF is further isolated and purified by using aqueous two phase extraction. To the diafiltered solution containing the refolded GCSF protein a phase forming polymer-salt combinations is added. Examples of phase forming agents, combinations of phase forming agents and parameters to consider in selecting suitable phase forming agents are discussed in Diamond et al., 1992, supra, and Abbott et al., 1990, Bioseparation 1:191-225, both of which are incorporated herein by reference in their entirety. The polymer and the salt are used under such conditions and at such concentrations so that a two-phase system is created.

Suitable polymers examples include but not limited to polyethylene glycol (PEG) or derivatives thereof having molecular weight of about 2000 to 8000 for example PEG 2000, PEG 4000, PEG 6000 and PEG 8000.

A phase forming salt includes inorganic or organic and preferably do not act to precipitate the polypeptide. Anions are selected that have the potential for forming aqueous multiple-phase systems. Examples include ammonium sulfate, sodium dibasic phosphate, sodium sulfate, ammonium phosphate, potassium citrate, magnesium phosphate, sodium phosphate, calcium phosphate, potassium phosphate, potassium sulfate, magnesium sulfate, calcium sulfate, sodium citrate, ammonium citrate, manganese sulfate, manganese phosphate, etc. Types of salts that are useful in forming bi-phasic aqueous systems are evaluated more fully in Zaslayskii et al., J. Chrom., 439: 267-281 (1988), which is incorporated herein by reference in its entirety. Preferred salts for the phase forming are sodium sulfate, potassium sulfate and ammonium sulfate.

In an embodiment of the invention the concentration of the phase forming agents may be varied. The concentration of the phase forming polymer, expressed in weight/volume is in the range of about 4% to about 18%, preferably from about 8% to about 12%. In yet another embodiment the concentration of the phase forming salt expressed in weight/volume is in the range of about 4% to about 18%, preferably from about 6% to about 12%.

The resulting extraction mixture is processed to form distinct phases, one of which contains an enrichment of the protein GCSF in the native form. Such processing can be accomplished, for example, by centrifuging the extraction mixture or by letting the mixture sit undisturbed for several hours (settle or coalesce at 1.times.gravity). In a further aspect, once distinct phases have been formed, the phase that contains an enrichment of the protein GCSF, i e., typically the upper light phase, may be removed.

Optionally, after removal of the phase that contains the protein GCSF, the phase that does not contain the protein GCSF may be reextracted (“two-stage extraction”). Reextraction can be performed by adding a solution containing a phase forming agent capable of forming a second light phase so that it will form a phase in the reextraction that is enriched in the protein GCSF. In another aspect, during two-stage extraction, the extraction mixture is stirred to dissolve the phase forming agents and to thoroughly mix the system. The resulting reextraction mixture is processed to form distinct phases of which one contains the enriched protein GCSF.

Following extraction purification, the protein GCSF can be detected in the phase removed from the extraction system. For example, the protein can be detected by a variety of methods including, but not limited to, bio assays, HPLC, amino acid determination or immunological assays, e.g., radioimmunoassay, ELISA, Western blot using antibody binding, SDS-PAGE. Such antibodies include but are not limited to polyclonal antibodies, monoclonal antibodies (mAbs), humanized or chimeric antibodies, single chain antibodies, Fab fragments, fragments produced by a Fab expression library, and epitope-binding fragments of any of the above. The amount of the purified protein and their level of purity can be determined by methods well known in the art.

The protein obtained using the method of the present invention can be further processed, for example, in order to provide the protein or polypeptide having high purity. Further purification may be necessary to remove related impurities. The impurities may include oxidized forms, deamidated forms, aggregated GCSF and also degraded forms such as biologically inactive monomeric forms, incorrectly folded molecules of G-CSF, denaturated forms of G-CSF, host cell proteins, host cell substances such as DNAs, (lipo)polysaccharides etc and additives which had been used in the preparation and processing of G-CSF. Such higher purity may be required depending on the use for which the protein or polypeptide is intended. For example, therapeutic uses of the protein will typically require further purification following the extraction methods of the invention. All protein purification methods known to the skilled artisan may be used for further purification. Such techniques have been extensively described in Berger and Kimmel, Guide to Molecular Cloning Techniques, Methods in Enzymology, Volume 152, Academic Press, San Diego, Calif. (1987); Molecular Cloning: A Laboratory Manual, 2d ed., Sambrook, J., Fritsch, E. F., and Maniatis, T. (1989); Current Protocols in Molecular Biology, John Wiley & Sons, all Viols., 1989, and periodic updates thereof); New Protein Techniques: Methods in Molecular Biology, Walker, J. M., ed., Humana Press, Clifton, N.J., 1988; and Protein Purification: Principles and Practice, 3rd. Ed., Scopes, R. K., Springer-Verlag, New York, N.Y., 1987, the above are incorporated herein by references in its entirety. In general, techniques including, but not limited to, ammonium sulfate precipitation, centrifugation, ion exchange, reverse-phase chromatography, affinity chromatography, hydrophobic interaction chromatography may be used to further purify the protein.

In a preferred embodiment the upper phase containing the GCSF protein is diluted to adjust the conductivity in the range of 3 to 6 mS/cm, preferably in the range of 4 to 5 mS/cm. The pH is also adjusted in the range of 3-5.5 preferably in the range of 4 to 5. The resultant solution containing GCSF along with related impurities can be further purified to remove related impurities by using cation-exchange chromatography. In another option the upper phase can also be subjected to hydrophobic interaction chromatography by proper salt addition.

The yield of the pure protein GCSF obtained by the processes of the invention are in the range of 40 to 50%.

According to one embodiment of the invention the aqueous two phase extraction is useful for separating more than 95% of the host cell proteins, endotoxins and DNA from the refolded protein GCSF. The protein GCSF obtained by the processes of the invention has a purity 99% or more. The GCSF obtained by the processes of the invention have very low oxidative impurities. The presence of endotoxins in the pure GCSF obtained by the processes of the invention is less than 2IU/ml. The content of host cell protein in the pure GCSF is less than 20 ppm.

The purification of GCSF in native form comprising at least one step of aqueous two phase extraction process according to the invention can be used for the native GCSF obtained from any of the natural sources like mammalian tissues and blood. The described process is particularly suitable for the industrial production of GCSF.

The process of obtaining pure GCSF as described herein further comprises of forming the pure GCSF into a finished dosage form for clinical use.

The biologically active G-CSF obtained by the entire process for the purification and/or isolation of the present invention is suitable for the preparation of pharmaceutical composition, which comprises the therapeutically effective amount of biologically active G-CSF and one or more pharmaceutical excipients and is suitable for clinical use. The possibility of maintaining the active form of G-CSF in a short purification and isolation process contributes not only to an improved yield, but also to an improved purity and effectiveness of the biologically active G-CSF and the pharmaceutical composition containing it.

Suitable pharmaceutically acceptable excipients include but not limited to suitable diluents, adjuvants and/or carriers useful in G-CSF therapy.

In yet another embodiment the invention relates to pharmaceutical compositions containing the GCSF obtained according to the present invention. The GCSF obtained can either be stored in the form of a lyophilisate or in liquid form. It is administered either subcutaneously or intravenously. Suitable adjuvants in the formulations of the recombinantly expressed GCSF are, for example, stabilizers like sugar and sugar alcohols, amino acids and tensides like for example polysorbate 20/80 as well as suitable buffer substances. Examples for formulations are described in EP 0674525, EP 0373679 and EP 0306824 both of which are incorporated herein by reference in its entirety.

The following examples are provided to further illustrate the present invention but are not provided to in any way limit the scope of the current invention.

### Example-1

### General Method for Obtaining Pure GCSF

Step A: Inclusion bodies of GCSF are solubilized in buffer containing 100 mM Tris 6M GuHCl pH 8.0. Solubilization takes around 45 min.The OD of the solubilized IB is adjusted with solubilization buffer to 8.0. (Generally 45 ml solubilization buffer for 1 g of IB is used). The solution is filtered through 0.45 μm filter. DTT is added up to 5 mM to reduce the protein. Reduction is carried out for 30 min at room temperature (25° C.).

Step B: The solubilized GCSF is added to refolding buffer with stirring in a period of 30-45 minutes. Refolding buffer contains 75 mM Tris pH 8.8, 0.1M L-Arginine, 10% Sucrose, 2 mM EDTA, 10 mM Sodium ascorbate, 2M Urea. For 1 g of IB 1 liter of refolding buffer is used. The temperature of the buffer is maintained at around 8.0° C. Refolding is carried out for 15-20 hrs. When sodium ascorbate is used in refolding buffer dehydro ascorbate and reduced glutathione are also added in refolding buffer to provide redox condition while refolding. Alternately, oxido-shuffling agents such as Cysteine/Cystine or Oxidised and reduced glutathione can also be used.

After refolding is over, buffer exchange of the refolded protein is carried out in 20 mM Tris pH 8.0, 5% Sucrose or 5% D+Sorbitol to remove the denaturant.

Step C: To the diafiltered solution containing the protein, PEG 4000 is added such that its concentration in the final solution would be 10% w/w. After the PEG is dissolved salt (Sodium sulfate) is added such that its concentration in the final solution would be 8% w/w. After the salt is dissolved the solution is left without stirring so that phase formation will take place. Two phases are formed, namely, Salt Phase and PEG Phase. The GCSF comes in the upper phase (PEG Phase). The lower phase is discarded, where impurities get removed. The upper phase is checked for purity. The pH of the upper phase, which contains GCSF protein, is adjusted to 4.5 and then either diafiltered or diluted to bring the conductivity to around 4-6 mS/cm.

Step D: This solution is then loaded on a cation exchanger (SP FF Sepahrose) at pH 4.5. The column is pre-equilibrated with 20 mM sodium acetate buffer pH 4.5. After loading is over the column is washed with 20 mM sodium acetate pH 5.5 buffer. After washing is over the bound protein is eluted with a linear gradient of NaCl in 20 mM sodium acetate pH 5.5 buffer.

Step E: The purified GCSF was then buffer exchanged into formulation buffer (10 mM sodium acetate, pH 4.0, 5% sorbitol, 0.004% Tween 80)

The purified GCSF protein is similar in-vitro bioactivity as the available commercial GCSF product.

### Example-2

2 g of inclusion bodies were solubilzed in 100 mM Tris pH 8.0, 6M Guanadium hydrochloride buffer. Solubilization was carried out at 25° C. and for 45 min. The solubilized IBs solution was filtered through 0.45 micron Polyether sulfone filter. The OD at 280 nm of the filtered solution was checked and adjusted to 8.0 by adding the required amount of solubilization buffer. To 90 ml of solubilized IB solution DTT was added such that the final concentration is 5 mM. Reduction was carried out for 30 min. After reduction the IB solution was slowly added to the 2000 ml refolding buffer with following composition: 75 mM Tris-Cl pH 8.8, 10% Sucrose, 2M Urea, 0.1M L-Arginine, 2 mM EDTA. The temperature was maintained at 8-10C. After the inclusion body solution is added cystine and cysteine are added such that the final concentration is 1 mM and 4 mM respectively. The refolding was carried out for 15 hrs at 10° C.

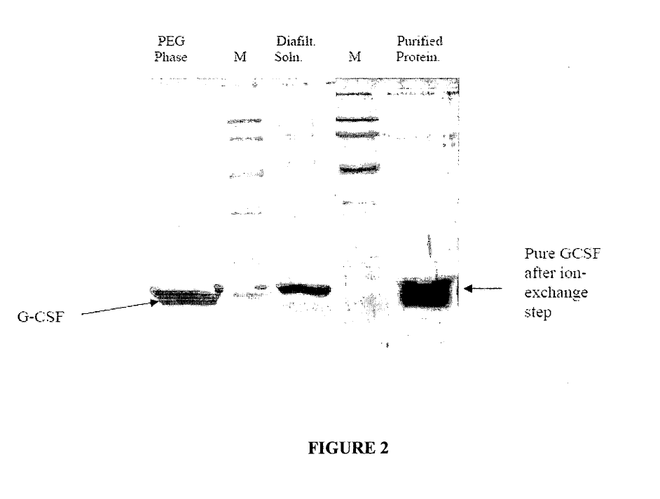

After the refolding was over the refolded protein was concentrated to 1 litre and diafiltered against 3 diafiltration volume of 20 mM Tris pH 8.0, 5% sorbitol using Tangential Flow Filtration (TFF). To the diafiltered solution 122 g of PEG 4000 was added. After the PEG was dissolved 97.6 g of sodium sulfate was added. The solution was then left for gravity settling. The upper phase was then recovered and diluted with 20 mM sodium acetate pH 4.5, 5% Sorbitol to adjust the pH to 4.5 and conductivity to 5.5 mS/cm. This diluted solution was then loaded on SP Sepharose column equilibrated with 20 mM sodium acetate pH 4.5, 5% sorbitol. After loading and washing with 20 mM sodium acetate pH 4.5 5% sorbitol buffer a further wash of 20 mM sodium acetate pH 5.5, 5% sorbitol buffer is provided. The bound protein was then eluted with a linear gradient of 20 mM sodium acetate pH 5.5, 5% sorbitol, 1M NaCl in 70CV. The eluted fractions containing purity more than 99% by RP-HPLC were pooled. The pooled fractions were then buffer exchanged against 10 mM sodium acetate, pH 4.0, 5% sorbitol, 0.004% Tween 80 using Sephadex G-25 medium gel filtration column. 200 mg of therapeutic grade Pure GCSF was obtained from the above process. FIG. 2 provides the SDS-PAGE profile of the Purification. The upper phase shows purity of more than 99% by SDS-PAGE. The final purified protein after ion-exchange shows purity more than 99% by SDS-PAGE and more than 98.5% by RP-HPLC. The yield obtained was 45%.

While the present invention has been described in terms of its specific embodiments, certain modifications and equivalents will be apparent to those skilled in the art and are intended to be included within the scope of the present invention.

1. A process for the preparation of pure recombinant human G-CSF obtained from microbial cells, the process comprising the steps of:
a) solubilizing one or more inclusion bodies of GCSF to obtain a solubilized GCSF protein;
b) refolding the solubilized GCSF protein to obtain a refolded GCSF protein;
c) purifying the refolded GCSF protein by using an aqueous two phase extraction; and
d) isolating the GCSF protein obtained in step c).

2. The process as claimed in claim 1, wherein the aqueous two phase extraction system comprises a phase forming polymer and a salt phase.

3. The process as claimed in claim 2, wherein the phase forming polymer comprises polyethylene glycol (PEG) at a molecular weight of about 2000 to about 8000.

4. The process as claimed in claim 2, wherein the salt phase comprises one or more of sodium sulfate, potassium sulfate and ammonium sulfate, sodium citrate, potassium citrate, ammonium citrate, sodium phosphate, ammonium phosphate and potassium phosphate.

5. The process as claimed in claim 2, wherein the concentration of the phase forming polymer is in the range of about 4% to about 18% w/v.

6. The process as claimed in claim 2, wherein the concentration of the phase forming salt is in the range of about 4% to about 18% w/v.

7. The process as claimed in claim 1, wherein the GSF protein obtained in step c) is further purified by a chromatographic purification step comprising one or more of ion exchange chromatography, reverse phase chromatography, affinity chromatography, hydrophobic interaction chromatography.

8. The process as claimed in claim 1, further comprising processing the GCSF protein obtained from step d) into a finished dosage form.

9. Pure G-CSF having purity of 99% or more, having endotoxins less than 2IU/ml and host cell protein less than 20 ppm.

10. Pure GCSF prepared by a process comprising at least one step of aqueous two phase extraction.

11. A pharmaceutical composition comprising a therapeutically effective amount of biologically active GCSF obtained by a process comprising at least one step of aqueous two phase extraction and one or more pharmaceutically acceptable excipients.
